(19) United States
(12) Patent Application Publication
(10) Pub. No.: US 2015/0328245 A1
(43) Pub. Date: Nov. 19, 2015

(54) Agents for Eliminating Tumour-Initiating Cells

(71) Applicant: GODAVARI BIOREFINERIES LIMITED, Mumbai, Maharashtra (IN)

(72) Inventors: Sangeeta Srivastava, Mumbai (IN); Annette Martin, Mumbai (IN); Maithili Athavale, Mumbai (IN); Kedar Shukre, Mumbai (IN)

(73) Assignee: GODAVARI BIOREFINERIES LIMITED, Mumbai (IN)

(21) Appl. No.: 14/653,254

(22) PCT Filed: Dec. 18, 2013
(86) PCT No.: PCT/IN13/00779

§ 371 Date: Jun. 17, 2015

(30) Foreign Application Priority Data
Dec. 18, 2012 (IN) 1752/MUM/2012

(51) Int. Cl. A61K 31/7048; A61K 45/06; C07H 17/04

(52) U.S. Cl. CPC A61K 31/7048; C07H 17/04; A61K 45/06

(57) ABSTRACT

The present invention provides agents useful for eliminating tumour initiating cells, compositions thereof, uses thereof and methods of using the same.

### FIELD OF THE INVENTION

The present invention relates to agents for eliminating tumour initiating cells and uses thereof in eliminating tumor initiating cells. The present invention also relates to a method of eliminating tumour initiating cells.

### BACKGROUND OF THE INVENTION

Recent research studies and findings have shown that cancer is driven by tumour-initiating cells (TICs) more commonly known as cancer stem cells the same has recently attracted a great deal of attention. Tumour-initiating cells can be rather referred to as cancer stem like cells since the origin of such tumor initiating cells is not known. Nonetheless such tumor initiating cells are considered as promising novel cellular target for the treatment of haematopoietic and solid malignancies. Furthermore, it seems that tumour-initiating cells might be resistant to many conventional cancer therapies, which might explain the limitations of these agents in curing the human malignancies.

Within a tumor, the majority of tumor cells have limited ability to proliferate and differentiate into cells that constitute the bulk of the tumor mass. However, recent studies have shown that a small population of cells within tumors possess the ability to self-renew and proliferate and are thus able to maintain the tumor. These cells, which are called tumor-initiating cells (TICs) or cancer stem cells (CSCs) have been observed to share certain characteristics with normal stem cells, including a stem cell-like phenotype and function. This particular type of cells seems to be able to initiate and drive tumor growth in different hematological and solid tumors. Further, because of slow replication and capacity for expelling anti-tumor drugs, these cells are resistant to many conventional cancer therapies. Tumour-initiating cells may have an important role in cancer relapse following treatment and might therefore be causative of the incurable nature of metastatic cancer. Because most modern anti-tumor agents have been approved based on tumor response, i.e., reduction in the number of differentiated tumor cells which form the bulk of most tumors, agents preferentially active on tumour-initiating cells may have been missed. Upon therapeutic intervention, many cells of the tumour are induced into apoptosis and die, while some survive and give rise to ‘second-line’ tumours with acquired resistance to the ‘first-line’ treatment. ‘Second-line’ tumours with higher resistance to therapy make them very hard to eliminate vastly complicating further therapy and making the prognosis very grim (Zobalova, et al, In Tech “Drugs that Kill Cancer Stem-like Cells” 06-02-2012).

Hence, there is an immediate need for newer agents that have ability to eliminate or inhibit the proliferation of the tumour initiating cells.

### SUMMARY OF THE INVENTION

Accordingly, the present invention provides in a general aspect compounds having the general formula I or a pharmaceutically acceptable salt thereof, which can have the ability of eliminating tumour-initiating cells.

Compounds of general formula I:

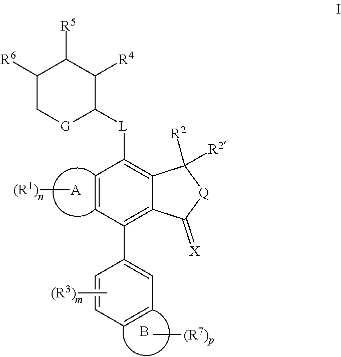

Each variable in Compounds of general formula I is further defined and described herein.

In a further aspect, the present invention provides compositions comprising compounds having the general formula I or a pharmaceutically acceptable salt thereof, for eliminating tumour-initiating cells.

In still further aspect the present invention provides a use of compounds having the general formula I or a pharmaceutically acceptable salt thereof or compositions thereof for eliminating tumour-initiating cells and thereby treating associated disorders or diseases or conditions.

In another aspect the present invention provides a method of eliminating tumour-initiating cells by administering compounds having the general formula I or a pharmaceutically acceptable salt thereof or compositions comprising the same in subjects in the needthereof.

### BRIEF DESCRIPTION OF THE DRAWINGS

FIG. 1 is a graph showing comparative results of in vitro 3D sphere forming stem cell assay of cisplatin (known chemotherapeutic agent) and compound I-2 for breast cancer cells MDA MB 231.

FIG. 2 is a graph showing comparative results of in vitro 3D sphere forming stem cell assay of cisplatin (known chemotherapeutic agent) and compound I-2 for prostate cancer cells PC3.

FIG. 3 is a graph showing comparative results of in vitro 3D sphere forming stem cell assay of cisplatin (known chemotherapeutic agent) and compound I-2 for prostate cancer cells LNCaP.

FIG. 4 is a graph showing comparative results of in vitro 3D sphere forming stem cell assay of cisplatin (known chemotherapeutic agent) and compound I-2 for breast cancer cells T47D.

FIG. 5 is a graph showing comparative results of in vitro 3D sphere forming stem cell assay of cisplatin (known chemotherapeutic agent) and compound I-2 for prostate cancer cells DU145.

FIG. 6 is a graph showing comparative results of in vitro 3D sphere forming stem cell assay of cisplatin (known chemotherapeutic agent) and compound I-3 for breast cancer cells MDA MB 231.

FIG. 7 is a graph showing comparative results of in vitro 3D sphere forming stem cell assay of cisplatin (known chemotherapeutic agent) and compound I-3 for breast cancer cells MCF-7.

### DESCRIPTION OF THE INVENTION

As used herein the term ‘tumour-initiating cells’ (TICS) refers to cells associated with or found in hematological and solid tumors. The term ‘tumour-initiating cells’ (TICS) may be used interchangeably with the term ‘cancer stem cells’ (CSCs). It will be appreciated by persons skilled in the art that the term ‘cancer stem cells’ (CSCs) would also refer to the term “cancer stem like cells.

The present invention provides compounds having, the general formula I or a pharmaceutically acceptable salt thereof, which can have ability of eliminating tumour-initiating cells.

Compounds of general formula I:

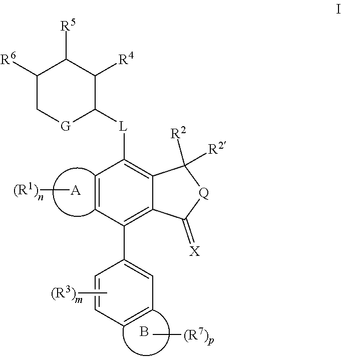

or a pharmaceutically acceptable salt thereof, wherein:
each of Ring A and Ring B is independently a 4-8 membered partially unsaturated, or aromatic ring having 0-3 heteroatoms independently selected from nitrogen, oxygen, or sulfur;
n is 0-4;
m is 0-3;
p is 0-2;
each R¹, R³ and R⁷ is independently halogen, —CN, —NO₂, —R, —OR, —SR, —N(R)₂, —N(R)C(O)R, —C(O)RN(R)₂, —N(R)C(O)N(R)₂, —N(R)C(O)OR, —OC(O)N(R), —N(R)SO₂R, —SO₂RN(R)₂, —C(O)R, —C(O)OR, —OC(O)R, —C(O)OR, —S(O)R, or —SO₂R;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each of R² and R^{2′} is independently hydrogen or an optionally substituted C₁₋₆ aliphatic, or: R² and R^{2′} are taken together to form ═O or ═S;

### Q is —O—, —S—, or —N(R)—;

### X is O or S;

L is a covalent bond or an optionally substituted bivalent C₁₋₆ hydrocarbon chain, wherein one methylene unit of L is optionally and independently replaced by —O—, —S—, —N(R)—, —C(O)—, —C(S)—, —C(O)N(R)—, —N(R)C(O)N(R)—, —N(R)C(O)—, —N(R)C(O)O—, —OC(O)N(R)—, —S(O)—, —S(O)₂—, —S(O)₂N(R)—, —N(R)S(O)₂—, —OC(O)— or —C(O)O—;

### G is —O—, —S—, or —N(R)—; and

each of R⁴, R⁵, and R⁶ is independently —OH, —OR, —OC(O)R, or a protected hydroxyl group.

As defined generally above, Ring A is a 4-8 membered partially unsaturated, or aromatic ring having 0-3 heteroatoms independently selected from nitrogen, oxygen, or sulfur. In certain embodiments, Ring A is a 5-6 membered partially unsaturated, or aromatic ring having 0-3 heteroatoms independently selected from nitrogen, oxygen, or sulfur. In some embodiments, Ring A is a 5-6 membered aromatic ring having 1-3 heteroatoms independently selected from nitrogen, oxygen, or sulfur. In some embodiments, Ring A is pyrido or thiopheno. In some embodiments, Ring A is benzo.

As defined generally above, Ring B is a 4-8 membered partially unsaturated or aromatic ring having 0-3 heteroatoms independently selected from nitrogen, oxygen, or sulfur. In some embodiments, Ring B is a 5-6 membered partially unsaturated or aromatic ring having 1-2 heteroatoms independently selected from nitrogen or oxygen. In some embodiments, Ring B is a 5 membered partially unsaturated or aromatic ring having 2 heteroatoms selected from nitrogen, oxygen or sulfur. In some embodiments, Ring B is 1,3-dioxolano.

As defined generally above, n is 0-4. In certain embodiments, n is 1-3. In some embodiments, n is 2. In some embodiments, n is 0.

As defined generally above, m is 0-3. In certain embodiments, m is 1-2. In some embodiments, m is 0.

As defined generally above, p is 0-2. In certain embodiments, p is 0. In some embodiments, p is 2.

As defined generally above, each R¹, R³ and R⁷ is independently halogen, —CN, —NO₂, —R, —OR, —SR, —N(R)₂, —N(R)C(O)R, —C(O)RN(R)₂, —N(R)C(O)N(R)₂, —N(R)C(O)OR, —OC(O)N(R), —N(R)SO₂R, —SO₂RN(R)₂, —C(O)R, —C(O)OR, —OC(O)R, —C(O)OR, —S(O)R, or —SO₂R. In certain embodiments, each R¹, R³ and R⁷ is independently halogen, —CN, —OR, —SR, or —OC(O)R. In some embodiments, each R¹ is independently halogen, —OR, or —OC(O)R. In some embodiments each R¹ is —OR. In some embodiments, each R³ is independently halogen or —OR. In some embodiments, each R⁷ is independently halogen, —C(O)OR, or —R. In some embodiments, each R⁷ is halogen. In some embodiments, each R⁷ is —R. In some embodiments, each R¹, R³ and R⁷ is hydrogen.

As defined generally above, each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In certain embodiments, each R is independently hydrogen or an optionally substituted group selected from C₁₋₆ aliphatic, phenyl, or a 0.5-6 membered monocyclic heteroaromatic ring having 1-4 heteroatoms independently selected from nitrogen, oxygen, or sulfur. In some embodiments, each R is independently hydrogen or an optionally substituted C₁₋₆ aliphatic group. In some embodiments, each R is independently hydrogen or an optionally substituted C₁₋₂ aliphatic group. In some embodiments, each R is hydrogen. In some embodiments, each R is an optionally substituted C₁₋₂ aliphatic group.

As defined generally above, each of R² and R^{2′} is independently hydrogen or an optionally substituted C₁₋₆ aliphatic, or: R² and R^{2′} are taken together to form ═O or ═S. In certain embodiments, each of R² and R^{2′} is independently hydrogen or R² and R^{2′} are taken together to form ═O or ═S. In some embodiments, each R² and R^{2′} is hydrogen. In some embodiments, R² and R^{2′} are taken together to form ═O or ═S.

As defined generally above, Q is —O—, —S—, or —N(R)—. In certain embodiments, Q is —O—, or —N(R)—. In some embodiments Q is —O—. In some embodiments, Q is —N(R)—.

As defined generally above, X is O or S. In certain embodiments, X is O. In some embodiments X is S.

As defined generally above, L is a covalent bond or an optionally substituted bivalent C₁₋₆ hydrocarbon chain, wherein one methylene unit of L is optionally and independently replaced by —O—, —S—, —N(R)—, —C(O)—, —C(S)—, —C(O)N(R)—, —N(R)C(O)N(R)—, —N(R)C(O)—, —N(R)C(O)O—, —OC(O)N(R)—, —S(O)—, —S(O)₂—, —S(O)₂N(R)—, —N(R)S(O)₂—, —OC(O)— or —C(O)O—. In certain embodiments, L is a covalent bond, or an optionally substituted bivalent C₁₋₆ hydrocarbon chain, wherein one methylene unit of L is optionally and independently replaced by —O—, —S—, or —N(R)—. In some embodiments, L is a covalent bond. In some embodiments, L is an optionally substituted bivalent C₁₋₂ hydrocarbon chain, wherein one methylene unit of L is optionally and independently replaced by —O—. In some embodiments, L is —O—.

As defined generally above, G is —O—, —S—, or —N(R)—. In certain embodiments, G is —O— or —N(R)—. In some embodiments G is —O—.

As defined generally above, each of R⁴, R⁵, and R⁶ is independently —OH, —OR, —OC(O)R, or a protected hydroxyl group. In certain embodiments, each of R⁴, R⁵, and R⁶ is independently —OH, —OR, or —OC(O)R. In some embodiments, R⁴ is —OH and R⁵, and R⁶ are —OR. In some embodiments, R⁴ is OC(O)R and R⁵, and R⁶ are —OR. In some embodiments, R⁴ is a protected hydroxyl group and R⁵, and R⁶ are —OR.

Hydroxyl protecting groups are well known in the art, examples of suitably protected hydroxyl groups of the R⁴, R⁵, and R⁶ groups of formula I further include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl.

Compounds of the present invention include those described generally above, and are further illustrated by the classes, subclasses, and species disclosed herein. Various terms and terminology used hereinabove in describing the compounds of the present invention and all technical and scientific terms used herein have the same would mean or refer to standard definition or meaning or as used in a chemical or technical field or as known or commonly understood by one of ordinary skill in the art to which this invention belongs.

Compounds of the present invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘}N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Exemplary pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate; nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms.

In certain embodiments, the present invention provides a compound of anyone of the formulas II-VIII:

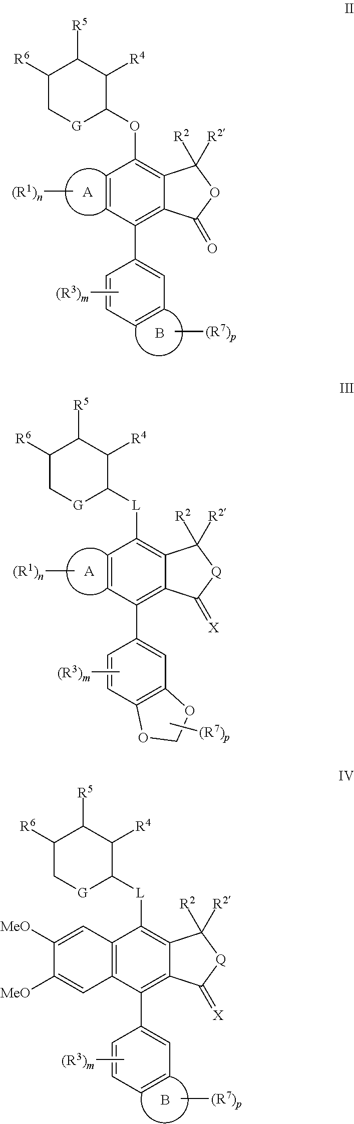

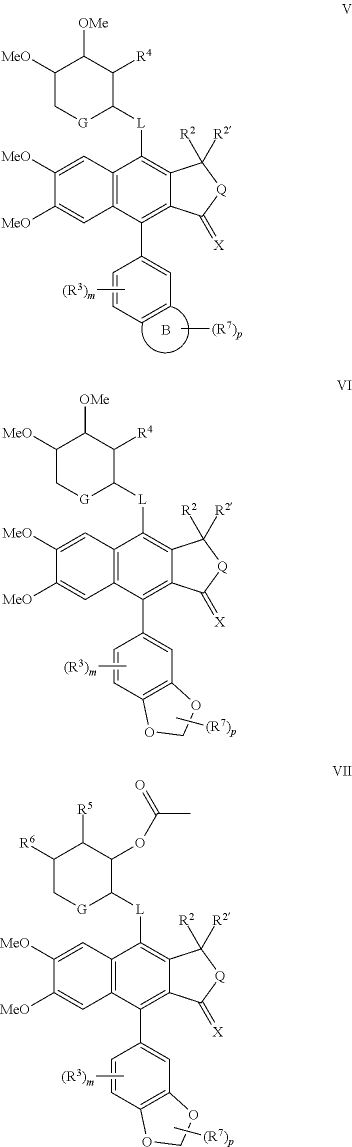

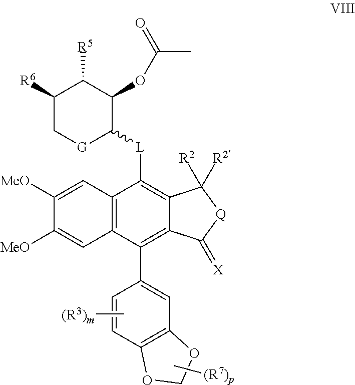

or a pharmaceutically acceptable salt thereof, wherein each variable is defined above and in classes and subclasses described above and herein for compounds of formula I.

In certain embodiments, the present invention provides a compound of the formula II, III, IV, V, VI, VII or VIII or a pharmaceutically acceptable salt thereof, wherein each variable is defined above and in classes and subclasses described above and herein for compounds of formula I.

In certain embodiments, the present invention provides a compound of formula I, wherein said compound is not selected from I-1 or I-2.

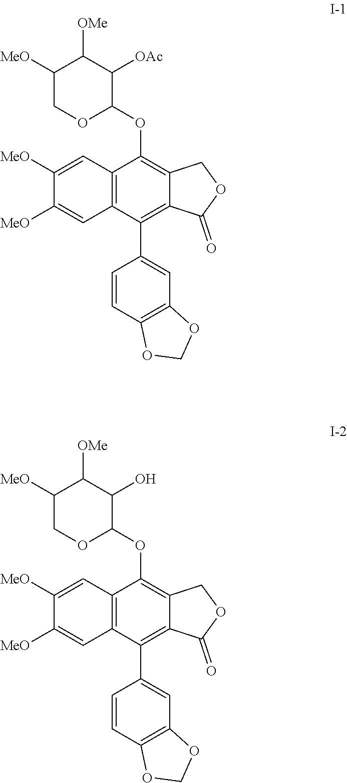 [SMILES: COc1cc2c(OC3OCC(OC)C(OC)C3OC(C)=O)c3c(c(-c4ccc5c(c4)OCO5)c2cc1OC)C(=O)OC3 ; COc1cc2c(OC3OCC(OC)C(OC)C3O)c3c(c(-c4ccc5c(c4)OCO5)c2cc1OC)C(=O)OC3]

In certain embodiments, the present invention provides any compound selected from those depicted above, or a pharmaceutically acceptable salt thereof.

In a preferred embodiment, the present invention provides a compound of the formula I-3:

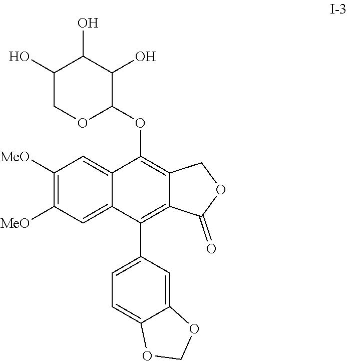 [SMILES: COc1cc2c(OC3OCC(O)C(O)C3O)c3c(c(-c4ccc5c(c4)OCO5)c2cc1OC)C(=O)OC3]

or a pharmaceutically acceptable salt thereof.

According to another embodiment, the invention provides a composition comprising a compound of this invention or a pharmaceutically acceptable salt or derivative thereof and a pharmaceutically acceptable carrier, adjuvant, or vehicle.

In certain embodiments, a composition of this invention comprises a compound having the general formula I, preferably the compound I-1, I-2 or I-3 or a pharmaceutically acceptable salt or derivative thereof and a pharmaceutically acceptable carrier, adjuvant, or vehicle.

In certain embodiments, a composition of this invention comprises a compound having anyone of the formulas II-VIII or a pharmaceutically acceptable salt or derivative thereof and a pharmaceutically acceptable carrier, adjuvant, or vehicle. The preferable derivative may be a pharmaceutically acceptable ester, or salt of an ester.

The amount of compound in compositions of this invention is such that is effective for eliminating or inhibiting the proliferation of TICs, in a biological sample or in a subject in the need thereof. In certain embodiments, the amount of compound in compositions of this invention is such that is effective to measurably eliminate TICs, in a biological sample or in a subject in the need thereof. In certain embodiments, the composition comprises between the biologically effective dose and the maximum tolerated dose of the compound of the invention or it's pharmaceutically acceptable salt, ester, or salt of an ester.

In certain embodiments, a composition of this invention is formulated for administration to a subject in the need thereof. In some embodiments, preferably a composition of this invention is formulated for oral administration to a patient. A “subject” is a mammal, preferably a human, but can also be an animal in need of veterinary treatment. The term “subject in the need thereof” refers to a patient suffering from disease, disorder or condition associated with proliferation of tumour initiating cells for example any type of cancer or relapse or recurrence of cancer.

The term “pharmaceutically acceptable carrier, adjuvant, or vehicle” refers to a non-toxic carrier, adjuvant, or vehicle that does not destroy the pharmacological activity of the compound with which it is formulated.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be formulated into a suitable dosage form to be administered orally, parenterally, by inhalation spray, topically, rectally, nasally, buccally, vaginally or via an implanted reservoir. Compositions of the present invention may be formulated into dosage forms including liquid, solid, and semisolid dosage forms. The term “parenteral” as used herein includes subcutaneous, intravenous, intraperitoneal, intramuscular, intra-articular, intra-synovial, intrasternal, intrathecal, intrahepatic, intralesional and intracranial injection or infusion techniques. Preferably, the compositions are administered orally, intravenously or intraperitoneally.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed include water, Ringer's solution and isotonic sodium chloride solution.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. Depot injectable formulations may also be prepared by entrapping the compound in liposomes or microemulsions that are compatible with body tissues.

Pharmaceutically acceptable compositions of this invention may be orally administered in any orally acceptable dosage form including, but not limited to, capsules, tablets, aqueous suspensions or solutions.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Ophthalmic formulation, ear drops, and eye drops are also contemplated as being within the scope of this invention. For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Topical application for the lower intestinal tract can be effected in a rectal suppository formulation (see above) or in a suitable enema formulation. Topically-transdermal patches may also be used. Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Most preferably, pharmaceutically acceptable compositions of this invention are formulated for oral administration. Such formulations may be administered with or without food.

The amount of compounds of the present invention that may be combined with the carrier materials to produce a composition in a single dosage form will vary depending upon the subject to be treated, the particular mode of administration. Preferably, provided compositions should be formulated so that a dosage of between 0.01-100 mg/kg body weight/day of the compound of the invention can be administered to a subject receiving these compositions. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will, also depend upon the particular compound in the composition.

In one embodiment the present invention provides a use of compounds having the general formula I or a pharmaceutically acceptable salt thereof or compositions thereof for eliminating tumour-initiating cells and thereby treating associated disorders or diseases or conditions. Thus, provided compounds are useful, for treating cancers, including, but not limited to hematological cancers and solid tumors.

In certain embodiments the present invention provides a use of compounds selected from I-1, I-2 or I-3 or a pharmaceutically acceptable salt thereof or compositions thereof for eliminating tumour-initiating cells and thereby treating associated disorders or diseases or conditions. Thus, provided compounds are useful for treating cancers, including, but not limited to hematological cancers and solid tumors.

In certain embodiments, the present invention provides a use of compounds having the formula II, III, IV, V, VI, VII or VIII or a pharmaceutically acceptable salt or derivative thereof or compositions thereof for eliminating tumour-initiating cells and thereby treating associated disorders or diseases or conditions. Thus, provided compounds are useful for treating cancers, including, but not limited to hematological cancers and solid tumors.

As used herein, the terms “eliminating tumour-initiating cells” refer to the elimination of the tumour-initiating cells by inhibiting or suppressing growth, division, maturation or viability of tumour-initiating cells, and/or causing the death of tumour-initiating cells, individually or in aggregate with other tumour-initiating cells; by cytotoxicity or the induction of apoptosis. One of skill in the art will appreciate that tumour-initiating cells are cancer cells, and by definition, “eliminating tumour-initiating cells” also encompasses the inhibition of the growth, division, maturation or viability of cancer cells, and/or causing the death of cancer cells, individually or in aggregate with other cancer cells, by cytotoxicity or the induction of apoptosis.

In another embodiment the present invention provides a method of eliminating tumour-initiating cells by administering compounds having the general formula I or a pharmaceutically acceptable salt thereof or compositions comprising the same in subjects in the needthereof.

In certain embodiments the present invention provides a method of eliminating tumour-initiating cells by administering the compound selected from I-1, I-2 or I-3 or a pharmaceutically acceptable salt thereof or compositions comprising the same in subjects in the needthereof.

In certain embodiments, the present invention provides a method of eliminating tumour-initiating cells by administering the compounds having the formula II, III, IV, V, VI, VII or VIII or a pharmaceutically acceptable salt or compositions comprising the same in subjects in the needthereof.

The activity of a compound utilized in this invention for eliminating tumour-initiating cells or other cancer cells, may be assayed in vitro or in vivo. An in vivo assessment of the eliminating or cytotoxic activity of the compounds of the invention may be made using an animal model of cancer, e.g., a rodent or primate model. Cell-based assays may be performed using, e.g., a cell line isolated from a tumor or blood-borne cancer. Cell-based assays for activity against a specific protein or nucleic acid component of a cancer cell line. e.g., an enzyme, structural protein, cell surface markers, DNA or RNA, or microarrays, may also be performed. Additionally, biochemical or mechanism-based assays, e.g., transcription assays using a purified protein, Northern blot, RT-PCR, etc., may be performed. In vitro assays include assays that determine cell morphology, viability, cell count, or growth inhibition, and/or the cytotoxicity, enzyme inhibitory activity, and/or the subsequent functional consequences of treatment of cancer cells with compounds of the invention. Alternate in vitro assays quantitate the ability of the compounds of the present invention to bind to protein or nucleic acid molecules within the cell. The aforementioned assays are exemplary and not intended to limit the scope of the invention. The skilled practitioner can appreciate that modifications can be made to conventional assays to develop equivalent assays that obtain the same result.

Examples of cancer cell lines that are inhibited by the compounds and compositions described herein and against which the methods described herein are useful include but are not limited to DU145, PC3, LNCaP, MDA MB 231, MCF7, T47D, HeLa, or other cell lines derived from tissues including, but not limited to, breast, prostate, colon, pancreas or lung. Inhibition of such cancer cells and cancer cell lines is in comparison with normal cells or cell lines that exhibit no inhibition or comparatively less inhibition by the compounds and compositions described herein.

According to one embodiment, the invention relates to a method of eliminating tumour-initiating cells in a biological sample comprising the step of contacting said biological sample with a compound of this invention, or a composition comprising said compound. In certain embodiments, the invention relates to a method of killing cancer cells or tumour-initiating cells in a biological sample comprising the step of contacting said biological sample with a compound of this invention, or a composition comprising said compound.

The term “compound of this invention” or “compound of the invention”, as used herein, includes the compounds having the general formula I, or compounds having the formula II, III, IV, V, VI, VII or VIII, or compounds I-1, I-2, or I-3 or a pharmaceutically acceptable salt thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Eliminating tumour-initiating cells in a biological sample is useful for a variety of purposes that are known to one of skill in the art. Examples of such purposes include, but are not limited to biological assays, gene expression studies, and biological target identification.

In one more embodiment the present invention provides a method of treatment of disorders or diseases or conditions associated with tumour-initiating cells by administering compounds of this invention or compositions comprising the same in subjects in the needthereof. According to certain embodiments, the invention relates to a method of eliminating tumour-initiating cells in a patient, leading to remission of the cancer, comprising the step of administering to said patient a compound of this invention or a composition comprising said compound. In other embodiments, the present invention provides a method for treating a disorder mediated by cancer cells or tumour-initiating cells, in a patient in need thereof, comprising the step of administering to said patient a compounds of this invention or pharmaceutically acceptable composition thereof. Such disorders are described in detail herein.

In some embodiments the compounds and compositions of the present invention may be used in a method of treating a cancer or other proliferative disorder. In some embodiments the present invention provides a method of treating a cancer or other proliferative disorder, comprising administering a compound or composition of the present invention to a patient with a cancer or other proliferative disorder. In certain embodiments the compounds and compositions of the present invention may be used to treat a cancer in a mammal. In certain embodiments the mammal is a human patient. In certain embodiments the compounds and compositions of the present invention may be used to treat a cancer in a human patient, said cancer occurring in the patient's breast, prostate, brain, blood, bone marrow, liver, pancreas, skin, kidney, colon, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, or stomach.

Depending upon the particular condition, or disease, to be treated, additional therapeutic agents, which are normally administered to treat that condition, may be administered in combination with compounds and compositions of this invention. In some embodiments, a provided compound of this invention, or composition thereof, is administered in combination with one or more other chemotherapeutic agents. Such chemotherapeutic agents include, but are not limited to agents such as kinase inhibitors, alkylating agents, anti-metabolites, tubulin stabilizers, tubulin assembly inhibitors, DNA replication inhibitors, cell cycle inhibitors, topoisomerase inhibitors, cytotoxic antibiotics or nanoparticle or protein conjugates of any of the aforementioned agents.

In certain embodiments, a combination of 2 or more chemotherapeutic agents may be administered together with compounds of the invention. In certain embodiments, a combination of 3 or more chemotherapeutic agents may be administered with compounds of the invention. In some embodiments, the chemotherapeutic agents are selected from alkylating agents or anti-metabolites.

Other examples of agents compounds of this invention may also be combined with include, without limitation: vitamins and nutritional supplements, cancer vaccines, other agents for treatments of conditions, disorders or diseases other than cancer for example neutropenia (e.g. G-CSF, filgrastim, lenograstim), treatments for thrombocytopenia (e.g. blood transfusion, erythropoietin), antiemetics (e.g. 5-HT₃ receptor antagonists, dopamine antagonists, NK1 receptor antagonists, histamine receptor antagonists, cannabinoids, benzodiazepines, or anticholinergics), treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

In certain embodiments, compounds of the invention, or a pharmaceutically acceptable composition thereof, are administered in combination with antisense agents, a monoclonal or polyclonal antibody or an siRNA therapeutic.

Those additional agents may be administered separately from the compound of the invention-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another. The amount of both, the compound of this invention and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Resistance to chemotherapeutic drugs is a major factor limiting the efficacy of therapies against many cancers and other proliferative disorders. The rapid division rate of these cells allows for the development of mutations or upregulation of pumps such as MDR that afford resistance to current first line chemotherapy drugs. The problem of relapse of cancers in a more drug-resistant form is a critical hurdle faced in drug development of new chemotherapeutic drugs to treat cancer patients.

The present invention addresses this problem by providing the compounds of this invention and compositions thereof for eliminating tumour-initiating cells and thereby treating associated disorders or diseases or conditions in particular for avoiding or minimizing problem of relapse of cancers.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

In certain embodiments, compounds of formula I where L, Q, and X are oxygen are prepared according to the procedure outlined in Scheme 1.

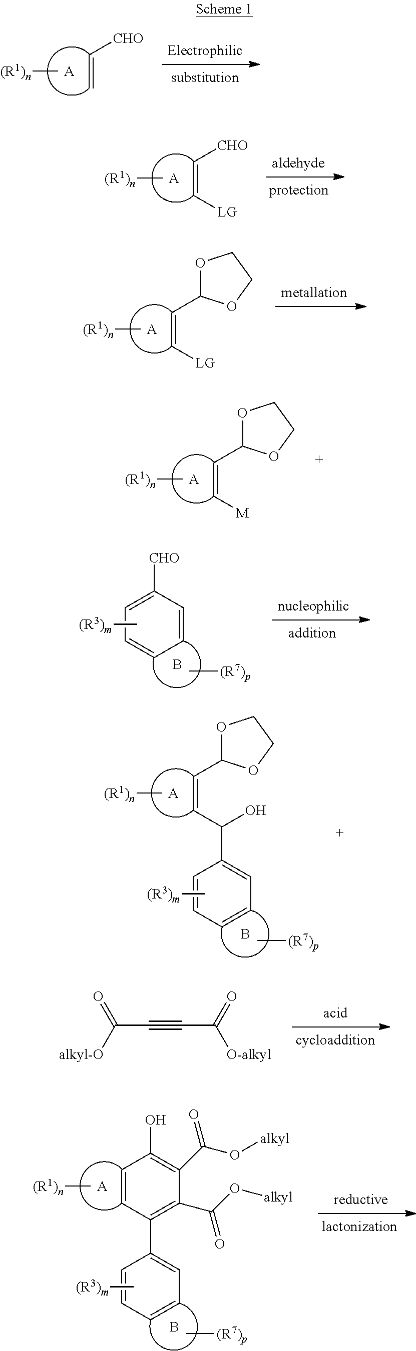

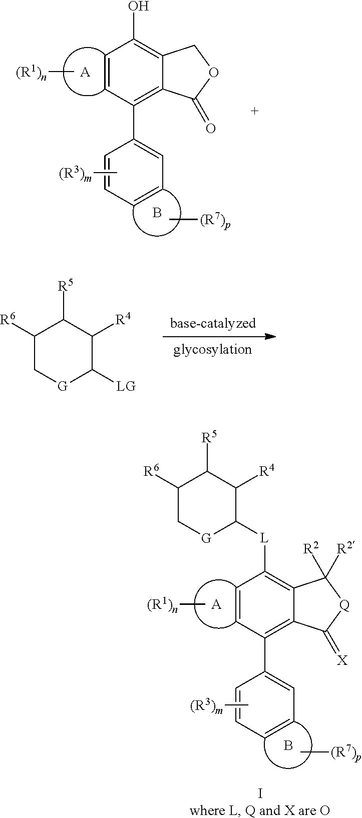

In certain embodiments, compounds of the invention and synthetic intermediates, thereof may be prepared according to methods known to one of ordinary skill in the art, including those described in WO2010/089778, WO2013/001352, the entirety of which are hereby incorporated herein by reference.

In certain embodiments, compounds of the invention are assayed as compounds eliminating tumour-initiating cells using methods known in the art including an in vitro cell death assay, an in vitro soft agar growth inhibition assay, an in vitro 3D sphere forming stem cell assay, and in vivo toxicity assays in animals. In some embodiments, assay results for compounds of the invention are compared to results obtained for known chemotherapeutic agents. In some embodiments, the known chemotherapeutic agent used for comparison is cisplatin.

While this invention has been particularly shown and described with references to various embodiments, it will be understood by those skilled in the art that various changes in form and details may be made therein without departing from the spirit and scope of the invention.

### EXAMPLES

To illustrate the invention, the following Examples are included. However, it is to be understood that these Examples do not limit the invention.

### Example 1

### Synthesis of 9-(1,3-benzodioxol-5-yl)-4-[(3,4,5-trihydroxytetrahydro-2H-pyran-2-yl)oxy]naphtho-6,7-dimethoxynaphtho[2,3-c]furan-1(3H)-one

Sealed tube was charged with (2-(1,3-Dioxolane-2-yl)-4,5-dimethoxy phenyl) (benzo(d)(1,3)dioxol-5-yl)-methanol (0.30 g, 0.833 mmole), diethyl acetylinedicarboxylate (0.141 g, 0.833 mole), dichloromethane (0.4 mL) and glacial acetic acid (0.242 mL) and mixture was heated at 140° C. for 1 hour. After completion of reaction as judged by TLC (50:50, EtOAc:Hexane), reaction mixture was cooled to room temperature, diluted with dichloromethane (10 mL), washed with 5% sodium bicarbonate solution (3×10 mL), organic layer was dried over anhydrous sodium sulfate, filtered and concentrated. The crude reaction mass was purified by flash column chromatography over silica gel using EtOAc:hexane (15:85) to afford diethyl 1-(3′,4′-methylenedioxyphenyl)-4-hydroxy-6,7-dimethoxynaphthalene-2,3-dicarboxylate as white solid 0.3 g (75%).

Two necked round bottom flask was charged with lithium aluminium hydride (0.032 g, 0.852 mmol) and anhydrous tetrahydrofuran (4 mL) and the mixture was cooled to 0° C. with stirring. To this suspension, a solution of diethyl 1-(3′,4′-methylenedioxyphenyl)-4-hydroxy-6,7-dimethoxynaphthalene-2,3-dicarboxylate (0.200 g, 0.426 mmol) in tetrahydrofuran (4 mL) was added drop wise and stirring was continued for 2 hours. After completion of reaction as judged by TLC (1:9, MeOH:DCM), reaction mixture was quenched with saturated sodium sulfate solution and extracted with n-butanol (4×20 mL). Organic layer was dried over anhydrous sodium sulfate, filtered and concentrated under reduced pressure. The crude residue was purified by flash column chromatography over silica gel to give yellow solid 9-(3′,4′-Methylenedioxyphenyl)-4-hydroxy-6,7-dimethoxynaphtho[2,3-c]furan-1(3H)-one 0.07 g (85%).

To a flame dried three necked round bottom flask (250 mL), 9-(3′,4′-methylenedioxyphenyl)-4-hydroxy-6,7-dimethoxynaptho[2,3-c]furan-1(3H)-one compound (0.380 g 1 mmole), and chloroform (20 mL) were added and stirred to dissolve completely. Then 2-3,4 oxy triacetyl-D-bromoxylopyranose (0.508 g, 1.5 mmole), tetra butyl ammonium bromide (0.322 g, 1 mmole) and 0.1 M sodium hydroxide solution (20 mL) were added in above solution. Reaction mass was kept for digestion under stirring for 6 hours, at 40° C., conversion was checked by TLC (MeOH:MDC, 0.5:9.5) and the conversion if found unconverted then digestion continued till conversion was complete. After completion of the reaction, the reaction mass was washed with 1% sodium bicarbonate solution. After layer separation, chloroform was concentrated to give crude solid mass 0.510 gms (yield 80%) of 9-(1,3-benzodioxol-5-yl)-4-[(3,4,5-triyl triacetate-2H-pyran-2-yl)oxy]naphtho-6,7-dimethoxynaphtho[2,3-c]furan-1(3H)-one.

In a clean dry three necked round bottom flask cleistanthin A triacetate (0.510, 0.8 mmole), potassium carbonate catalytic amount (0.138 g, 1 mmole) and methanol 20 mL) were added & stirred well for 1 Hr at temperature 25° C. Reaction was monitored by TLC and HPLC. After completion of reaction, the reaction mass was quenched by water and extracted with ethyl acetate (2×30 mL) to get the final product. Ethyl acetate was concentrated to get pure 0.307 gm (yield 60%) of 9-(1,3-benzodioxol-5-yl)-4-[(3,4,5-trihydroxytetrahydro-2H-pyran-2-yl)oxy]naphtho-6,7-dimethoxynaphtho[2,3-c]furan-1(3H)-one.

The NMR details of the compound I-3 were as follows:

1HNMR (CDCl3,300 MHz): 2.1 ppm(t) 1H—OCH, 2.2 ppm(m) 1H—OCH, 3.4 ppm(m) 2H—OCH2, 3.6 ppm(m) 3H—OCH3, 3.8 ppm(q) 1H—OCH, 3.9 ppm(s) 3H—OCH3, 4.7 ppm(q) 1H—OCHO—, 5 ppm(d) 1H—OH, 5.2 ppm(m) 1H—OH, 5.4 ppm(d) 2H Ar—OCH2, 5.9 ppm(d) 1H—OH, 6.1 ppm(s) 2H—OCHO—, 6.8 ppm 1H Ar—H, 6.91 ppm(d) Ar—H, 6.92 ppm(d) Ar—H, 6.95 ppm(d) Ar—H, 7.01 ppm(d) Ar—H.

### Example 2

### In Vitro Colorimetric Cell Death Assay

It's an in vitro colorimetric assay for assessing cell viability where the cells are grown in Two-dimensional surface.

An exemplary procedure for the assay follows. Cancer cells and normal cells were plated in a 96 well plate as per predetermined plating efficiency. The plate was incubated for 24 hours in a 5% CO₂ atmosphere at 37 degrees Celsius, a range of concentrations of the compounds I-1, I-2 and I-3 as well as cisplatin (known chemotherapeutic agents) were added to the separate wells, the plate was incubated further for 48 hours in a 5% CO₂ atmosphere, the plate was centrifuged twice at 3000 rpm for 3 minutes, the supernatant fluid was discarded, 100 uL of 0.5 mg/mL MTT solution was added and the plate was incubated for 4 hours in a 5% CO₂ atmosphere at 37 degrees Celsius. The plate was then centrifuged twice at 3000 rpm for 3 minutes, supernatant, was aspirated very carefully, 200 uL DMSO was added to each well to solubilize MTT crystals and mixed well by shaking the plate, the plate was incubated for 10 minutes in a 5% CO₂ atmosphere at 37 degrees Celsius, the plate placed on the shaker of an ELISA plate reader and the absorbance at 570 nm was measured, then the percentage of viable cells remaining was calculated by first subtracting the background absorbance then comparing to the absorbance of a non-drug-treated cell sample, and the results were plotted on a graph to determine the IC50 for the compounds and cisplatin.

The results of the in vitro colorimetric cell death assay are set forth in Table 1, Table 2 and Table 3.

TABLE 1
Comparative results of in vitro colorimetric cell death assay
of cisplatin (known chemotherapeutic agent) and compound I-1:

| | Cisplatin | I-1 |
|---|---|---|
| Cell Lines | IC 50 (uM) | IC 50 (uM) |
| DU145 | 56.23 | 37.58 |
| L 929 | 46.7 | 1.23 |
| MCF10A (normal breast | 36.3 | No activity |
| cell line) | | |

TABLE 2
Comparative results of in vitro colorimetric cell death assay
of cisplatin (known chemotherapeutic agent) and compound I-2:

| | Cisplatin | I-2 |
|---|---|---|
| Cell Lines | IC 50 (uM) | IC 50 (uM) |
| MCF-7 | 4.750 | 0.9538 |
| MDAMB231 | 82.26 | 0.3305 |
| Du145 | 56.23 | 2.45 |
| L929 | 46.7 | 1.38 |
| MCF 10A (normal breast | 36.3 | No Activity |
| cell line) | | |

TABLE 3
Comparative results of in vitro colorimetric cell death assay
of cisplatin (known chemotherapeutic agent) and compound I-3:

| | Cisplatin | I-3 |
|---|---|---|
| Cell Lines | IC 50 (uM) | IC 50 (uM) |
| MDAMB 231 | 82.26 | 10 |
| T47D | 51.28 | 21.13 |
| PC3 | 40.06 | 13.5 |
| DU145 | 56.23 | 9.55 |
| LNCaP | 64.56 | 3.16 |

The compound I-1, I-2 and I-3 showed high anticancer activity in cancer cell lines as compared to cisplatin (known chemotherapeutic agent). However, the compounds did not show activity on the normal cell line whereas while cisplatin showed significant anti cancer activity.

### Example 3

### In Vitro Soft Agar Colony Forming Growth Assay

The Soft Agar Assay for Colony Formation is considered the most stringent assay for detecting malignant transformation of cells. In this assay the cells are cultured in medium with low percentage of agar.

An exemplary procedure for the assay follows. A mixture of 50 uL of 2× medium and 50 uL of 1.2% Bacto Agar was plated onto each well of a 96 well microtiter assay plate, 10 uL of cells (of specific plating efficiency pre-standardized per cell line) were mixed with 20 uL of 2× medium and 30 uL of 0.8% Bacto Agar and 1.6 uL of the compounds of the invention and cisplatin individually in a separate vial, the mixture was transferred to the solidified agar layer of each respective well of the plate, the plate was incubated at 37 degrees Celsius in 5% CO₂ for one week (feeding each well after 3 days with 50 uL of 2× medium), then 16 uL of Alamar Blue (1.5 mg/mL) was added to each well, the absorbance of each well was measured at 630 nm and percent viability of each well relative to the absorbance reading of the growth control well without compound and cisplatin was calculated, and the IC50 of the compound and cisplatin was determined.

Results of the in vitro soft agar assay are set forth in following Tables.

TABLE 4
Comparative results of in vitro soft agar colony forming growth assay
of cisplatin (known chemotherapeutic agent) and compound I-1:

| | Cisplatin | I-1 |
|---|---|---|
| Cell line | IC 50 (uM) | IC 50 (uM) |
| MDAMB231 | 10.96 | 5.88 |
| L929 | 8.7 | 3.63 |
| HeLa | 39.35 | 5.12 |

TABLE 5
Comparative results of in vitro soft agar colony forming growth assay
of cisplatin (known chemotherapeutic agent) and compound I-2:

| | Cisplatin | I-2 |
|---|---|---|
| Cell line | IC 50 (uM) | IC 50 (uM) |
| MCF-7 | 37.15 | 0.62 |
| MDAMB231 | 37.34 | 3.63 |
| T47D | 51.28 | 2.39 |
| DU145 | 56.23 | 3.16 |
| PC3 | 40.06 | 0.26 |
| LNCaP | 64.56 | 0.7 |

TABLE 6
Comparative results of in vitro soft agar colony forming growth assay
of cisplatin (known chemotherapeutic agent) and compound I-3:

| | Cisplatin | I-3 |
|---|---|---|
| Cell line | IC 50 (uM) | IC 50 (uM) |
| MDAMB231 | 37.34 | 0.55 |
| T47D | 51.28 | 0.7 |
| DU145 | 56.23 | 0.661 |
| PC3 | 40.06 | 0.85 |

The numbers of colonies formed by the cancer cells were significantly less in the compounds of the invention than those formed in cisplatin. Thus the compounds of the invention show high anticancer activity than cisplatin (known chemotherapeutic agent).

### Example 4

### In Vitro 3D Primary Sphere Forming Stem Cell Assay

This assay measures the ability of cancer stem cells to form spheres in a serum free media where the spheres are in a suspended form. Through this assay we evaluated the potency of the compounds of the invention to kill cancer stem cells in comparison with the known chemotherapeutic agent Cisplatin.

An exemplary procedure for the assay follows. Cells were grown in three dimensions on a plastic substrate, harvested in suspension in serum-free media, then the cells in the sample were trypsinized and a single cell suspension was formed by passing through a cell strainer. The cells were diluted according to the predetermined plating efficiency for the cell line being studied by suspending the cells in stem cell culture medium. 100 uL of this suspension was added into each well of a 96 well suspension plate, and the plate was incubated at 37 degrees Celsius in 5% CO₂ atmosphere for 24 hours, then 2 uL of appropriate concentrations of the compounds of the invention and cisplatin were added into each respective well along with 100 uL of stem cell culture medium, and the plates were incubated at 37 degrees Celsius under 5% CO₂ atmosphere for 72 hours. 2.5 uL of the compounds of the invention and cisplatin were added to each respective well along with 50 uL of stem cell culture medium and then the plates were incubated at 37 degrees Celsius under 5% CO₂ atmosphere for 72 hours. 3 uL of appropriate concentrations of the compounds of the invention and cisplatin were added to each respective well along with 50 uL of stem cell culture medium and then the plates were incubated at 37 degrees Celsius under 5% CO₂ atmosphere for 72 hours, then the spheres formed were observed under a microscope then counted and scored by size.

Results of the in vitro 3D sphere forming stem cell assay are set forth in a tabular manner. The number in each box is the total number of spheres formed in the presence of either cisplatin or compound I-2 or compound I-3 at each drug concentration. GC refers to a growth control performed in the absence of drug or solvent (DMSO). GCD refers to a growth control performed in the absence of drug, but in the presence of DMSO.

TABLE 7
Comparative results of in vitro 3D sphere forming stem cell assay of
cisplatin (known chemotherapeutic agent) and compound I-2 for breast cancer cells
MDA MB 231:
# of 3D Primary Spheres Formed of Cells MDA MB231 (±SD)

| | Dilution (from stock of 0.5M) | | | | | | |
|---|---|---|---|---|---|---|---|
| | 10 | 100 | 1000 | 10,000 | 100,000 | | |
| | Final conc. | | | | | | |
| | 1250 μM | 125 μM | 12.5 μM | 1.25 μM | 0.125 μM | GC | GCD |
| Cisplatin | 34(±10) | 103(±13) | 112(±18) | 113(±11) | 124(±7) | 116(±13) | 117(±12) |
| I-2 | 0(±0) | 0(±0) | 14(±2) | 32(±5) | 60(±9) | 116(±13) | 117(±12) |

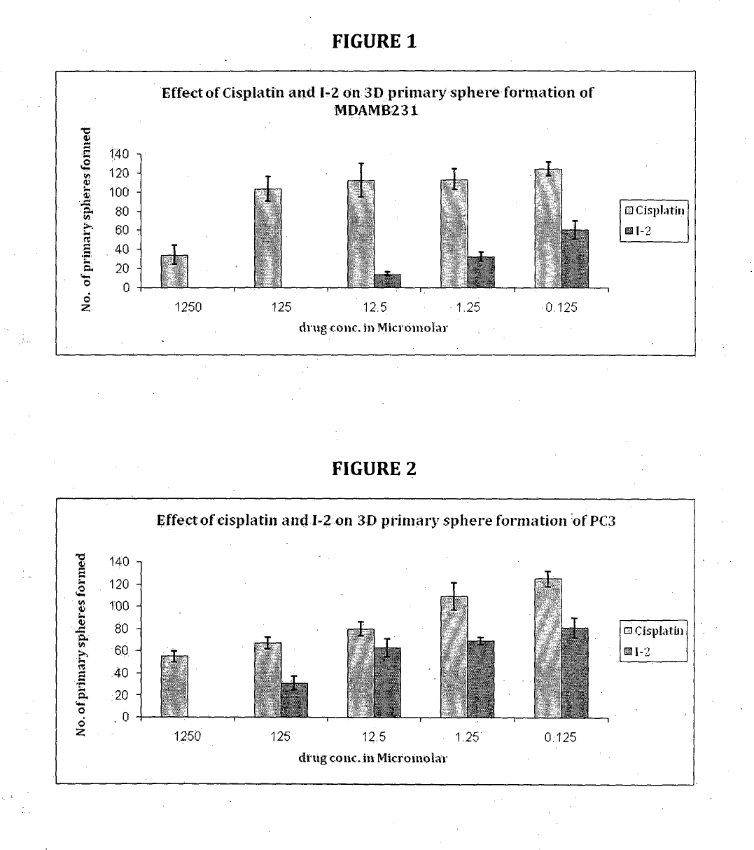

The numbers of primary spheres formed of the breast cancer cells (MDAMB 231) were significantly less in I-2 than those formed in cisplatin as can be seen in the above table as well as FIG. 1. Thus, the compound I-2 showed high anticancer activity than cisplatin (known chemotherapeutic agent).

TABLE 8
Comparative results of in vitro 3D sphere forming stem cell assay of
cisplatin (known chemotherapeutic agent) and compound I-2 for prostate cancer cells PC3:
# of 3D Primary Spheres Formed of Cells PC3 (±SD)

| | Dilution (from stock of 0.5M) | | | | | | |
|---|---|---|---|---|---|---|---|
| | 10 | 100 | 1000 | 10,000 | 100,000 | | |
| | Final conc. | | | | | | |
| | 1250 μM | 125 μM | 12.5 μM | 1.25 μM | 0.125 μM | GC | GCD |
| Cisplatin | 55(±5) | 67(±5) | 80(±6) | 109(±12) | 125(±7) | 139(±9) | 138(±8) |
| I-2 | 0(±0) | 31(±6) | 63(±8) | 69(±3) | 81(±9) | 139(±9) | 138(±8) |

The numbers of primary spheres formed of the prostrate cancer cells (PC3) were significantly less in I-2 than those formed in cisplatin as can be seen in the above table as well as FIG. 2. Thus, the compound I-2 showed high anticancer activity than cisplatin (known chemotherapeutic agent).

TABLE 9
Comparative results of in vitro 3D sphere forming stem cell assay of
cisplatin (known chemotherapeutic agent) and compound I-2 for prostate cancer cells LNCaP:
# of 3D Primary Spheres Formed of Cells LNCaP (±SD)

| | Dilution (from stock of 0.5M) | | | | | | |
|---|---|---|---|---|---|---|---|
| | 10 | 100 | 1000 | 10,000 | 100,000 | | |
| | Final conc. | | | | | | |
| | 1250 μM | 125 μM | 12.5 μM | 1.25 μM | 0.125 μM | GC | GCD |
| Cisplatin | 9(±2) | 54(±7) | 108(±10) | 144(±8) | 142(±12) | 149(±10) | 125(±8) |
| I-2 | 0(±0) | 0(±0) | 6(±2) | 49(±6) | 98(±17) | 149(±10) | 125(±8) |

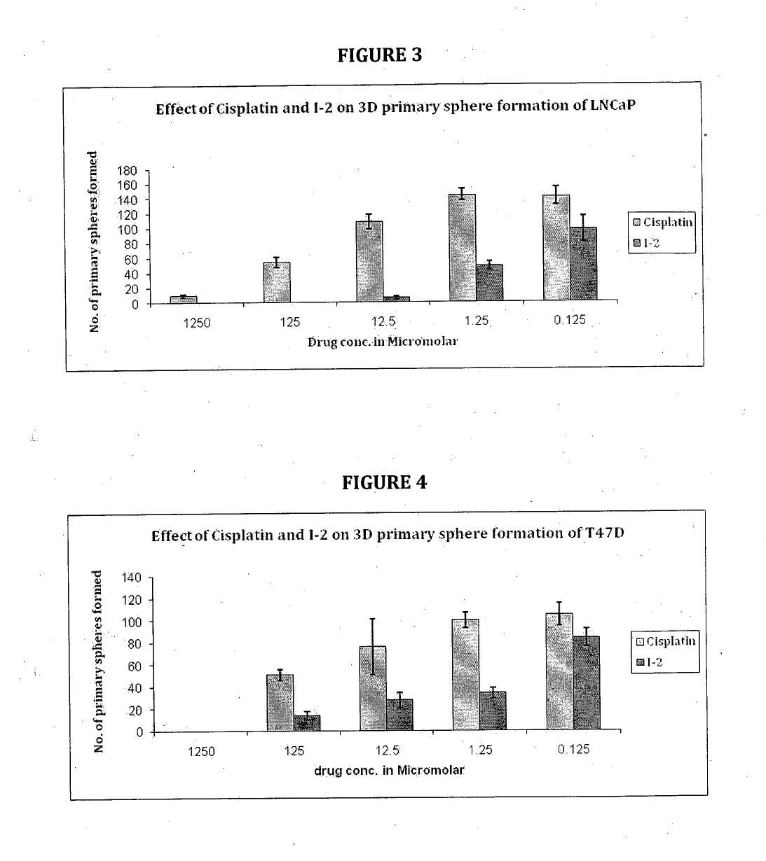

The numbers of primary spheres formed of the prostrate cancer cells (LNCaP) were significantly less in I-2 than those formed in cisplatin as can be seen in the above table as well as FIG. 3. Thus, the compound I-2 showed high anticancer activity than cisplatin (known chemotherapeutic agent).

TABLE 10
Comparative results of in vitro 3D sphere forming stem cell assay of
cisplatin (known chemotherapeutic agent) and compound I-2 for breast cancer cells
T47D:
# of 3D Primary Spheres Formed of Cells T47D (±SD)

| | Dilution (from stock of 0.5M) | | | | | | |
|---|---|---|---|---|---|---|---|
| | 10 | 100 | 1000 | 10,000 | 100,000 | | |
| | Final conc. | | | | | | |
| | 1250 μM | 125 μM | 12.5 μM | 1.25 μM | 0.125 μM | GC | GCD |
| Cisplatin | 0(±0) | 51(±5) | 76(±25) | 100(±7) | 105(±10) | 113(±9) | 101(±8) |
| I-2 | 0(±0) | 14(±4) | 28(±7) | 34(±5) | 84(±8) | 113(±9) | 101(±8) |

The numbers of primary spheres formed of the breast cancer cells T47D were significantly less in I-2 than those formed in cisplatin as can be seen in the above table as well as FIG. 4. Thus, the compound I-2 showed high anticancer activity than cisplatin (known chemotherapeutic agent).

TABLE 11
Comparative results of in vitro 3D sphere forming stem cell assay of
cisplatin (known chemotherapeutic agent) and compound I-2 for prostate cancer
cells DU145:
# of 3D Primary Spheres Formed of Cells DU145 (±SD)

| | Dilution (from stock of 0.5M) | | | | | | |
|---|---|---|---|---|---|---|---|
| | 10 | 100 | 1000 | 10,000 | 100,000 | | |
| | Final conc. | | | | | | |
| | 1250 μM | 125 μM | 12.5 μM | 1.25 μM | 0.125 μM | GC | GCD |
| Cisplatin | 11(±3) | 35(±4) | 48(±6) | 70(±7) | 84(±4) | 92(±8) | 85(±3) |
| I-2 | 0(±0) | 7(±1) | 18(±7) | 41(±4) | 53(±5) | 92(±8) | 85(±3) |

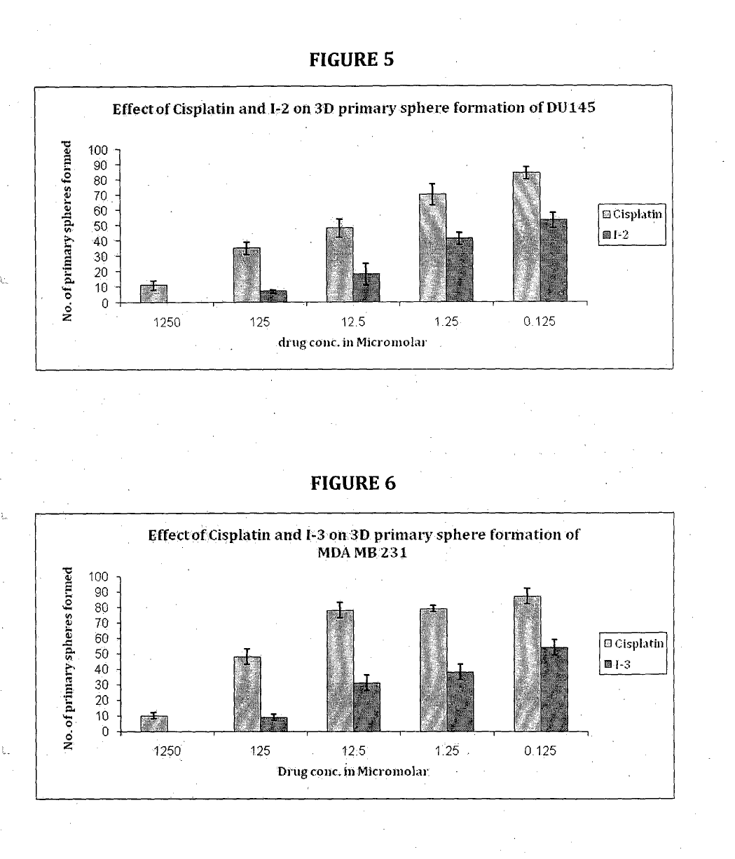

The numbers of primary spheres formed of the prostate cancer cells DU145 were significantly less in I-2 than those formed in cisplatin as can be seen in the above table as well as FIG. 5. Thus, the compound I-2 showed high anticancer activity than cisplatin (known chemotherapeutic agent).

TABLE 12
Comparative results of in vitro 3D sphere forming stem cell assay of
cisplatin (known chemotherapeutic agent) and compound I-3 for breast cancer cells
MDA MB 231:
# of 3D Primary Spheres Formed of Cells MDA MB 231 (±SD)

| | Dilution (from stock of 0.5M) | | | | | | |
|---|---|---|---|---|---|---|---|
| | 10 | 100 | 1000 | 10,000 | 100,000 | | |
| | Final conc. | | | | | | |
| | 1250 μM | 125 μM | 12.5 μM | 1.25 μM | 0.125 μM | GC | GCD |
| Cisplatin | 10(±2) | 48(±5) | 78(±5) | 79(±2) | 87(±5) | 80(±5) | 68(±3) |
| I-3 | 0(±0) | 9(±2) | 31(±5) | 38(±5) | 54(±5) | 80(±5) | 68(±3) |

The numbers of primary spheres formed of the breast cancer cells MDA MB 231 were significantly less in I-3 than those formed in cisplatin as can be seen in the above table as well as FIG. 6. Thus, the compound I-3 showed high anticancer activity than cisplatin (known chemotherapeutic agent).

TABLE 13
Comparative results of in vitro 3D sphere forming stem cell assay of
cisplatin (known chemotherapeutic agent) and compound I-3 for breast cancer cells
MCF-7:
# of 3D Primary Spheres Formed of Cells MCF-7 (±SD)

| | Dilution (from stock of 0.5M) | | | | | | |
|---|---|---|---|---|---|---|---|
| | 10 | 100 | 1000 | 10,000 | 100,000 | | |
| | Final conc. | | | | | | |
| | 1250 μM | 125 μM | 12.5 μM | 1.25 μM | 0.125 μM | GC | GCD |
| Cisplatin | 37(±8) | 61(±7) | 68(±7) | 77(±5) | 76(±5) | 77(±6) | 63(±5) |
| I-3 | 0(±0) | 28(±6) | 48(±10) | 57(±5) | 74(±10) | 80(±5) | 68(±3) |

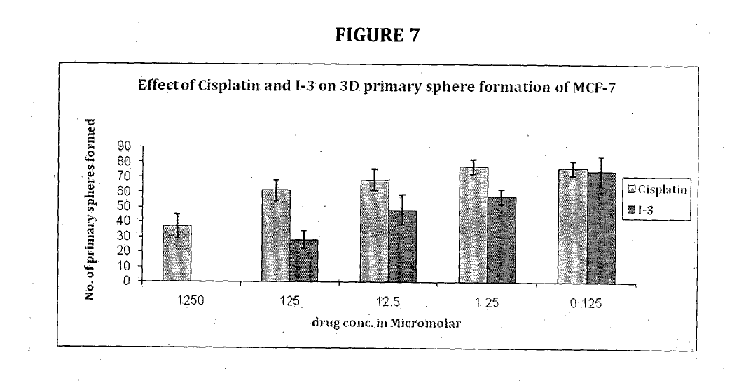

The numbers of primary spheres formed of the breast cancer cells MCF-7 were significantly less in I-3 than those formed in cisplatin as can be seen in the above table as well as FIG. 7. Thus, the compound I-3 showed high anticancer activity than cisplatin (known chemotherapeutic agent).

### Example 5

### In Vivo Maximum Tolerated Dose Assay for Compound of Invention I-1

In vivo maximum tolerated dose (MTD) toxicity assay was carried out to evaluate the highest dose of a pharmaceutical treatment that will produce the desired effect without unacceptable toxicity. The purpose of administering MTD is to determine whether long-term exposure to a compound might lead to unacceptable adverse health effects in a subject, when the level of exposure is not sufficient to cause premature mortality due to short-term toxic effects.

An exemplary procedure for the in vivo maximum tolerated dose toxicity assay follows. A statistically appropriate number of male and female nude mice were injected with a single dose of either 30 mg/kg, 10 mg/kg, 5 mg/kg, and 2.5 mg/kg of a compound I-1 interperitoneally (i.p.) on day zero. The number of mice surviving from each dose group after the first through fourth days was recorded, and the Maximum Tolerated Dose (“MTD”; e.g. the dose at which 100% of the animals survived after 4 days) was determined.

The results of the in vivo MTD assay for compound I-1 are set forth in Table 14.

TABLE 14
Results of MTD assay for compound I-1

| | Number of mice surviving after injection (time in days) | | | | |
|---|---|---|---|---|---|
| I-1 dose | 0 | 1 | 2 | 3 | 4 |
| 30 mg/kg | 4/6 | 4/6 | 0/6 | 0/6 | 0/6 |
| 10 mg/kg | 6/6 | 4/6 | 4/6 | 4/6 | 4/6 |
| 5 mg/kg | 6/6 | 6/6 | 6/6 | 6/6 | 6/6 |
| 2.5 mg/kg | 6/6 | 6/6 | 6/6 | 6/6 | 6/6 |

The dose that resulted in 100% survival of mice after 4 days was about 5 mg/kg of compound I-1 per kilogram of animal body weight, indicating that MTD for I-1 is in the range of 5-10 mg/kg body weight.

### Example 6

### In Vivo Maximum Tolerated Dose Assay for Compound of Invention I-2

In vivo maximum tolerated dose (MTD) toxicity assay was carried out to evaluate the highest dose of a pharmaceutical treatment that will produce the desired effect without unacceptable toxicity. The purpose of administering MTD is to determine whether long-term exposure to a compound might lead to unacceptable adverse health effects in a subject, when the level of exposure is not sufficient to cause premature mortality due to short-term toxic effects.

An exemplary procedure for the in vivo maximum tolerated dose toxicity assay follows. A statistically appropriate number of FEMALE BALB/c MICE were interperitoneally treated with the compound I-2 at three different dose levels 3 mg/kg, 1 mg/kg, and 0.3 mg/kg. Body weight and clinical sign of all the animals were observed daily during dosing and post dosing period.

The results of the in vivo MTD assay for compound I-2 are set forth in Table 15. It shows the Mean Body weight (Unit: g; Mean±SEM) of the FEMALE BALB/c MICE when injected with compound of invention I-2 with q7d×4 (weekly one dose for 4 weeks) at different dosage.

TABLE 15
Results of MTD assay for compound I-2

| | G1 I-2 3 mg/kg | | G2 I-2 1 mg/kg | | G3 I-2 0.3 mg/kg | |
|---|---|---|---|---|---|---|
| | q7dX4 | | q7dX4 | | q7dX4 | |
| Days | Mean (g) | SEM | Mean (g) | SEM | Mean (g) | SEM |
| 1 | 2 | 1.1 | 23. | 0.8 | 2 | 0.8 |
| 2 | 2 | 1.0 | 23. | 0.9 | 2 | 0.6 |
| 3 | 2 | 1.0 | 23. | 1.0 | 2 | 0.6 |
| 4 | 2 | 0.7 | 24. | 1.0 | 2 | 0.5 |
| 5 | 2 | 0.7 | 24. | 0.8 | 2 | 0.6 |
| 6 | 2 | 0.7 | 24. | 0.7 | 2 | 0.7 |
| 7 | 2 | 0.9 | 24. | 0.8 | 2 | 0.8 |
| 8 | 2 | 1.0 | 24. | 0.8 | 2 | 0.7 |
| 9 | 2 | 1.0 | 24. | 0.8 | 2 | 0.8 |
| 10 | 2 | 0.8 | 24. | 0.9 | 2 | 0.8 |
| 11 | 2 | 0.9 | 24. | 0.9 | 2 | 0.8 |
| 12 | 2 | 1.2 | 24. | 0.8 | 2 | 0.7 |
| 13 | 2 | 0.9 | 25. | 0.9 | 2 | 0.7 |
| 14 | 2 | 0.9 | 24. | 0.9 | 2 | 0.7 |
| 15 | 2 | 1.2 | 24. | 0.8 | 2 | 0.8 |
| 16 | 2 | 1.2 | 25. | 0.8 | 2 | 0.7 |
| 17 | 2 | 0.8 | 25. | 0.9 | 2 | 0.6 |
| 18 | 2 | 1.2 | 25. | 0.9 | 2 | 0.7 |
| 19 | 2 | 1.1 | 25. | 0.9 | 2 | 0.7 |
| 20 | 2 | 1.1 | 25. | 0.9 | 2 | 0.7 |
| 21 | 2 | 1.1 | 25. | 0.9 | 2 | 0.7 |
| 22 | 2 | 1.2 | 25. | 0.9 | 2 | 0.8 |
| 23 | 2 | 1.2 | 25. | 0.9 | 2 | 0.7 |
| 24 | 2 | 1.1 | 25. | 0.9 | 2 | 0.7 |
| 25 | 2 | 1.0 | 25. | 0.9 | 2 | 0.7 |
| 26 | 2 | 0.9 | 26. | 0.9 | 2 | 0.7 |
| 27 | 2 | 1.0 | 25. | 0.9 | 2 | 0.7 |
| 28 | 2 | 1.3 | 25. | 0.9 | 2 | 0.6 |
| 29 | 2 | 1.2 | 25. | 0.9 | 2 | 0.6 |

As can be seen from the above table, group G1 I-2 at dose 3 mg/kg and group G3 I-2 at dose 0.3 mg/kg showed decreased body weight after first treatment then they showed normal growth pattern. Group G3 showed marginal body weight gain as compared to group G1. Group G2 at dose I-2 1 mg/kg body weight showed normal growth pattern throughout the experimental period. G2 showed maximum body weight gain as compared to other treatment group. Percentage Body weight change in group G1 was observed at day 2 i.e −3.2% throughout the experimental period. Percentage Body weight change in group G3 was observed at day 3 i.e −3.8% throughout the experimental period. All the animals were observed daily after dosing and post dosing period for mortality and clinical signs. As more than one mortality at dose 3 mg/kg was observed so the given dose was found to be toxic. However, 1 mg/kg and 0.3 mg/kg found to be safe to the experimental animals at the given treatment regimen. Hence the MTD of I-2 will be less than 3 mg/kg and more than 1 mg/kg at the regimen of q7d×4.

While the invention has been described in conjunction with enumerated embodiments, it will be understood that they are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

What is claimed is:

1. A compound of formula I: 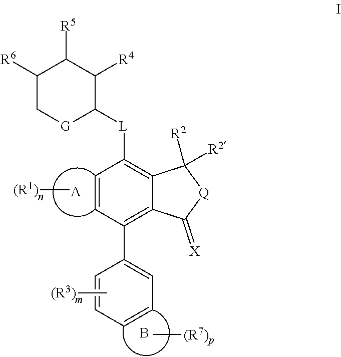
or a pharmaceutically acceptable salt thereof, wherein:
each of Ring A and Ring B is independently a 4-8 membered partially unsaturated, or aromatic ring having 0-3 heteroatoms independently selected from nitrogen, oxygen, or sulfur;
n is 0-4;
m is 0-3;
p is 0-2;
each R¹, R³ and R⁷ is independently halogen, —CN, —NO₂, —R, —OR, —SR, —N(R)₂, —N(R)C(O)R, —C(O)RN(R)₂, —N(R)C(O)N(R)₂, —N(R)C(O)OR, —OC(O)N(R), —N(R)SO₂R, —SO₂RN(R)₂, —C(O)R, —C(O)OR, —OC(O)R, —C(O)OR, —S(O)R, or —SO₂R;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each of R² and R² is independently hydrogen or an optionally substituted Q.
6 aliphatic, or: R² and R^{2′} are taken together to form ═O or ═S;
Q is —O—, —S—, or —N(R)—;
X is O or S;
L is a covalent bond or an optionally substituted bivalent C₁₋₆ hydrocarbon chain, wherein one methylene unit of L is optionally and independently replaced by —O—, —S—, —N(R)—, —C(O)—, —C(S)—, —C(O)N(R)—, —N(R)C(O)N(R)—, —N(R)C(O)—, —N(R)C(O)O—, —OC(O)N(R)—, —S(O)—, —S(O)₂— —S(O)₂N(R)—, —N(R)S(O)₂— —OC(O)— or —C(O)O—;
G is —O—, —S—, or —N(R)—; and
each of R⁴, R⁵, and R⁶ is independently —OH, —OR, —OC(O)R, or a protected hydroxyl group;
wherein the compound is not

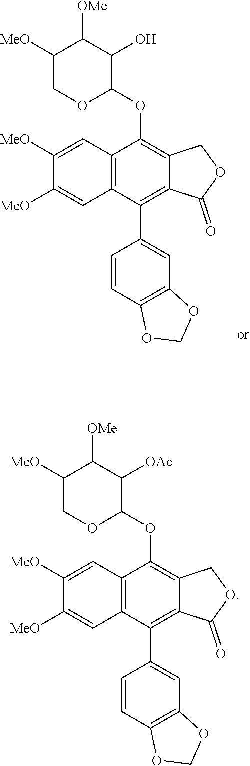 [SMILES: COc1cc2c(OC3OCC(OC)C(OC)C3O)c3c(c(-c4ccc5c(c4)OCO5)c2cc1OC)C(=O)OC3 ; COc1cc2c(OC3OCC(OC)C(OC)C3OC(C)=O)c3c(c(-c4ccc5c(c4)OCO5)c2cc1OC)C(=O)OC3]

2. The compound as claimed in claim 1, wherein the compound is selected from anyone of the compounds of formula II, III, IV, V, VI, VII VIII: 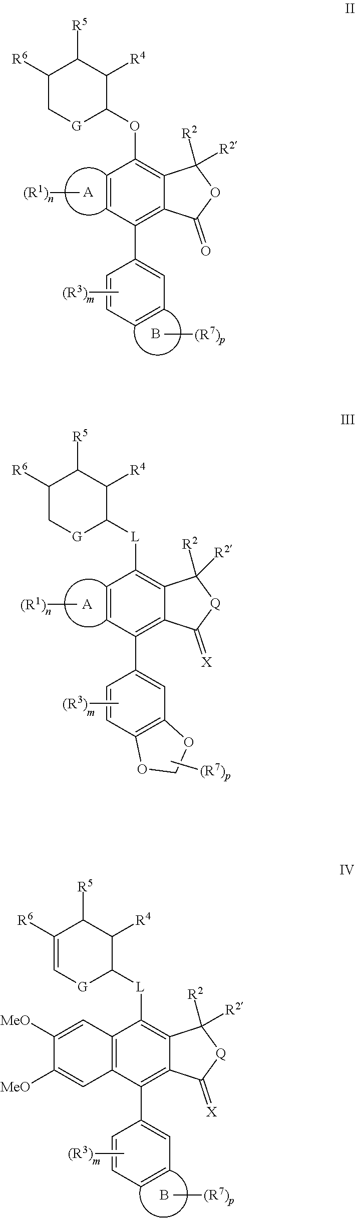 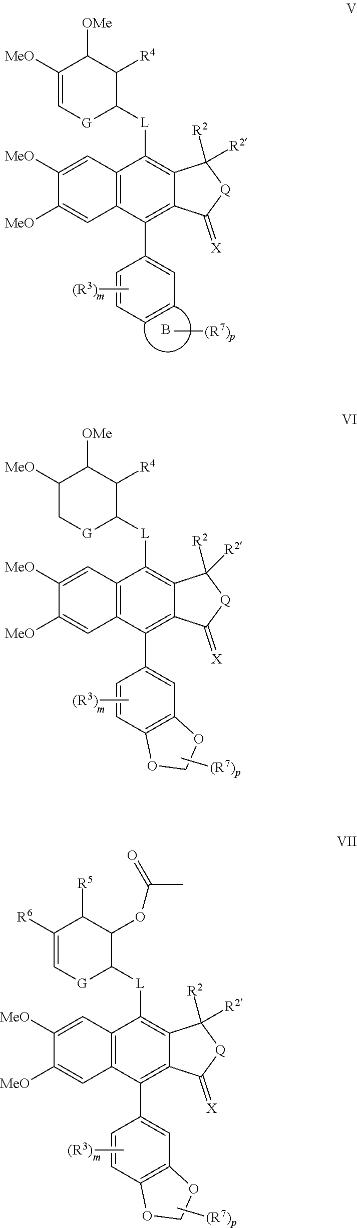 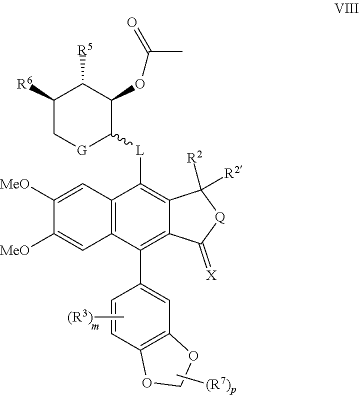
or a pharmaceutically acceptable salt thereof.

3. The compound according to claim 1, wherein the compound is:

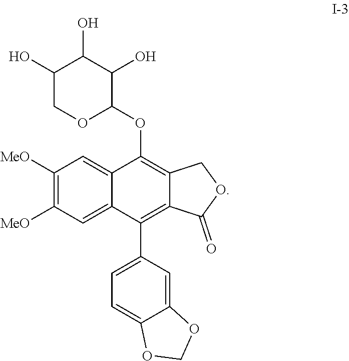 [SMILES: COc1cc2c(OC3OCC(O)C(O)C3O)c3c(c(-c4ccc5c(c4)OCO5)c2cc1OC)C(=O)OC3]

4. A composition comprising the compound according to claim 1 or a pharmaceutically acceptable salt, ester, or salt of an ester thereof, and a pharmaceutically acceptable carrier, adjuvant, or vehicle.

5. The composition according to claim 4, wherein the composition comprises between the biologically effective dose and the maximum tolerated dose of the compound of formula I, or it's pharmaceutically acceptable salt, ester, or salt of an ester.

6-8. (canceled)

9. A method of eliminating tumour-initiating cells in a patient in need thereof, comprising the step of administering to said patient a compound of formula I: 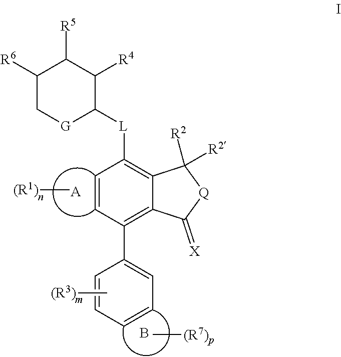
or a pharmaceutically acceptable salt thereof, wherein:
each of Ring A and Ring B is independently a 4-8 membered partially unsaturated, or aromatic ring having 0-3 heteroatoms independently selected from nitrogen, oxygen, or sulfur;
n is 0-4;
m is 0-3;
p is 0-2;
each R¹, R³ and R⁷ is independently halogen, —CN, —NO₂, —R, —OR, —SR, —N(R)₂, —N(R)C(O)R, —C(O)RN(R)₂, —N(R)C(O)N(R)₂, —N(R)C(O)OR, —OC(O)N(R), —N(R)SO₂R, —SO₂RN(R)₂, —C(O)R, —C(O)OR, —OC(O)R, —C(O)OR, —S(O)R, or —SO₂R; each R is independently hydrogen or an optionally substituted group selected from Ci-6 aliphatic, a 3-8 membered saturated or partially unsaturated monocyclic carbocyclic ring, phenyl, an 8-10 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each of R² and R^{2′} is independently hydrogen or an optionally substituted C₁₋₆ aliphatic, or: R² and R² are taken together to form ═O or ═S;
Q is —O—, —S—, or —N(R)—;
X is O or S;
L is a covalent bond or an optionally substituted bivalent C₁₋₆ hydrocarbon chain, wherein one methylene unit of L is optionally and independently replaced by -0-, —S—, —N(R)—, —C(O)—, —C(S)— —C(O)N(R)—, —N(R)C(O)N(R)—, —N(R)C(O)—, —N(R)C(O)0- —OC(O)N(R)—, —S(O)—, —S(O)₂— —S(O)₂N(R)—, —N(R)S(O)₂— —OC(O)— or —C(O)0-;
G is —O—, —S—, or —N(R)—; and
each of R⁴, R⁵, and R⁶ is independently —OH, —OR, —OC(O)R, or a protected hydroxyl group;
or a pharmaceutically acceptable salt thereof or compositions thereof, wherein the composition comprises between the biologically effective dose and the maximum tolerated dose of the compound of formula I or it's pharmaceutically acceptable salt, ester, or salt of an ester.

10. The method according to claim 9, wherein the compound is selected from anyone of the compounds of formula II, III, IV, V, VI, VII or VIII:

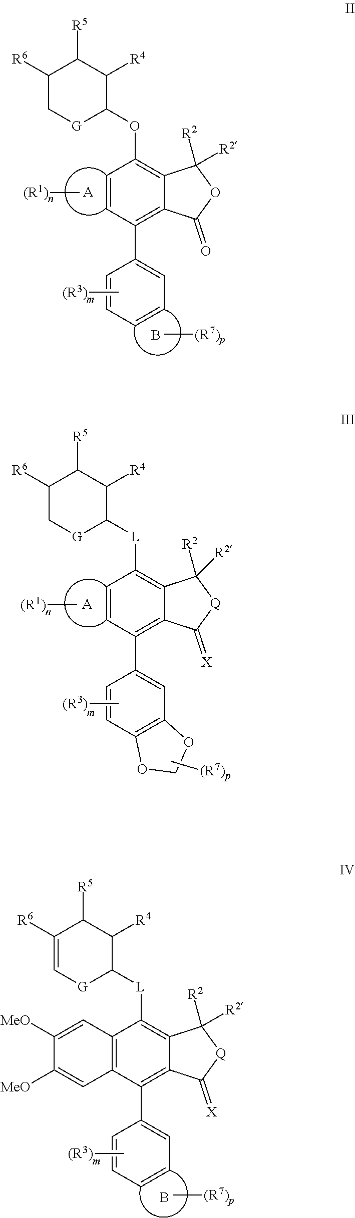

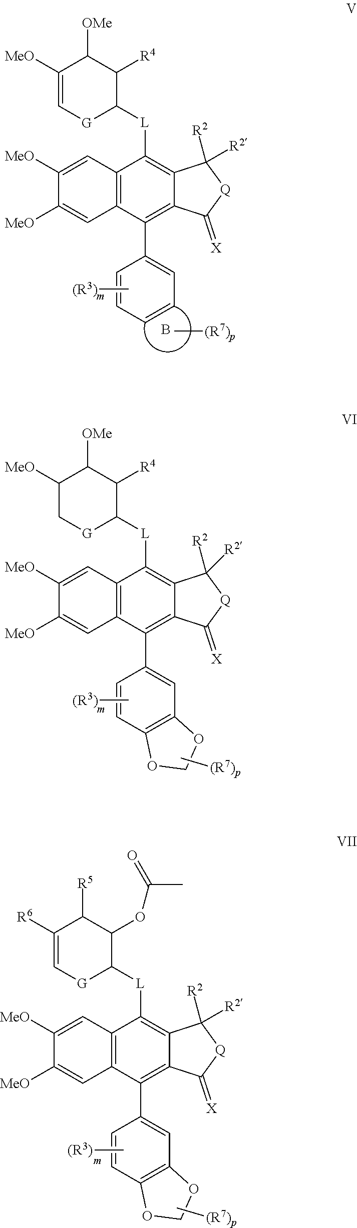

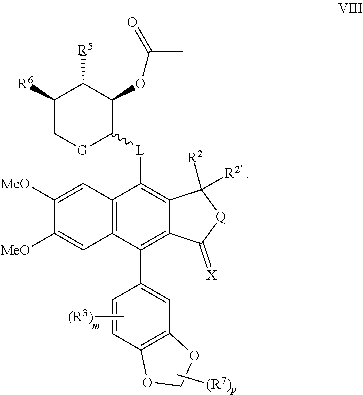

11. The method as claimed in claim 9, wherein the compound is anyone of compounds selected from 1-1, 1-2 or 1-3:

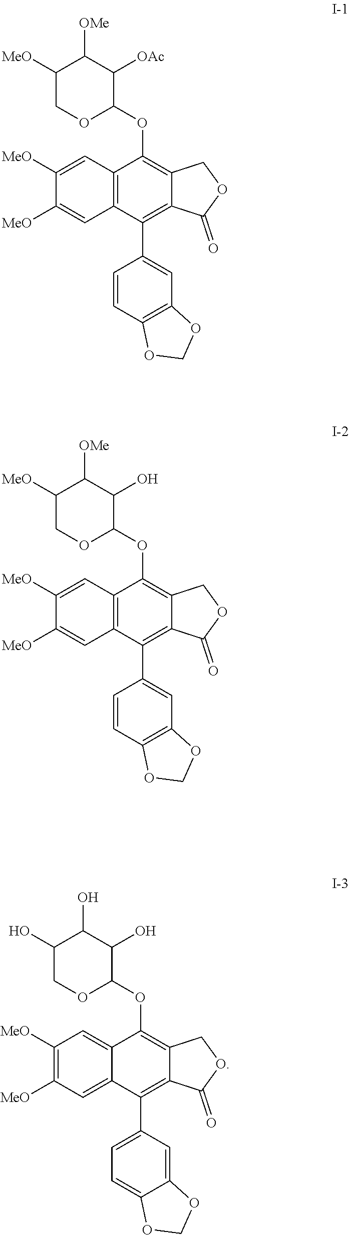 [SMILES: COc1cc2c(OC3OCC(OC)C(OC)C3OC(C)=O)c3c(c(-c4ccc5c(c4)OCO5)c2cc1OC)C(=O)OC3 ; COc1cc2c(OC3OCC(OC)C(OC)C3O)c3c(c(-c4ccc5c(c4)OCO5)c2cc1OC)C(=O)OC3 ; COc1cc2c(OC3OCC(O)C(O)C3O)c3c(c(-c4ccc5c(c4)OCO5)c2cc1OC)C(=O)OC3]

12. The method according to claim 9, wherein said cancer stem cells are found in either:
a) the following cell lines: DU145, PC3, LIMCaP, MDA MB 231, MCF7, T47D or HeLa; or
b) a cancer tissue sample obtained from breast, prostate, brain, blood, bone marrow, liver, pancreas, skin, kidney, colon, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart; or stomach.

13. The method according to claim 9, wherein said elimination leads to remission of a cancer.

14. The method according to claim 13, wherein said cancer is selected from breast, prostate, brain, blood, bone marrow, liver, pancreas, skin, kidney, colon, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, or stomach.

15. The method according to claim 9, wherein said compound or a pharmaceutically acceptable salt thereof is administered in combination with an additional chemotherapeutic agent.

16. The method according to claim 15, wherein said additional chemotherapeutic agent is selected from kinase inhibitors, alkylating agents, anti-metabolites, tubulin stabilizers, tubulin assembly inhibitors, DNA replication inhibitors, cell cycle inhibitors, topoisomerase inhibitors, cytotoxic antibiotics or nanoparticle or protein conjugates of any of the aforementioned agents. In certain embodiments, the chemotherapeutic agents used in combination with compounds or compositions of the invention include, but are not limited to imatinib, nilotinib, gefitinib, sunitinib, carfilzomib, salinosporamide A, retinoic acid, cisplatin, carboplatin, oxaliplatin, mechlorethamine, cyclophosphamide, chlorambucil, ifosfamide, azathioprine, mercaptopurine, doxifluridine, fluorouracil, gemcitabine, methotrexate, tioguanine, vincristine, vinblastine, vinorelbine, vindesine, podophyllotoxin, etoposide, teniposide, tafluposide, paclitaxel, docetaxel, irinotecan, topotecan, amsacrine, actinomycin, doxorubicin, daunorubicin, valrubicin, idarubicin, epirubicin, plicamycin, mitomycin, mitoxantrone, melphalan, busulfan, capecitabine, pemetrexed, epothilones, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Citrovorum Factor, Cladribine, Cortisone, Cosmegen®, CPT-11, Cytadren®, Cytosar-U®, Cytoxan®, Dacarbazine, Dacogen, Dactinomycin, Darbepoetin Alfa, Dasatinib, Daunomycin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, Ethyol, Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab, ozogamicin, Gemzar Gleevec™, Gliadel® Wafer, GM-CSF, Gosereliri, Granulocyte-Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab, Tiuxetan, Idamycin®, Idarubicin Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase®, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Me™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, Octreotide, Octreotide acetate, Ofatumumab, Oncospar®, Oncovin®, Ontak®, Onxal™, Oprelvekin, Orapred®, Orasone®, Oxaliplatin, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlinriid®, Rheumatrex®, Rituxan®, Rituximab, Roferon-A® (Interferon Alfa-2a), Romiplostim, Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisei®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, Xeloda®, Zanosar®, Zevalin™, Zinecard®, Zoladex®, Zoledronic acid, Zolinza, Zometa®, or combinations thereof.
